(19) United States
(12) Patent Application Publication
(10) Pub. No.: US 2013/0326793 A1
(43) Pub. Date: Dec. 12, 2013

(54) APPARATUS AND METHOD FOR WEARING REPLICA CAULIFLOWER EARS

(71) Applicants: Andrew James Bingley, Fergus (CA); Walter David McCrindle, Fergus (CA); Christopher Brian Prickett, Fergus (CA)

(72) Inventors: Andrew James Bingley, Fergus (CA); Walter David McCrindle, Fergus (CA); Christopher Brian Prickett, Fergus (CA)

(21) Appl. No.: 13/858,613

(22) Filed: Apr. 8, 2013

Related U.S. Application Data
(60) Provisional application No. 61/621,275, filed on Apr. 6, 2012.

(51) Int. Cl. A61F 11/00

(52) U.S. Cl. CPC A61F 11/00
USPC 2/209

(57) ABSTRACT

There is disclosed an apparatus and method for wearing replica “cauliflower” ears which are molded from the actual cauliflower ears of athletes contact sports including mixed martial arts (MMA), wrestling, rugby, boxing and others. In an embodiment, the apparatus comprises support means adapted to support left and right replica cauliflower ears on a wearer's head. A fastening means is adapted to fasten the left and right replica cauliflower ears, respectively, to the support means. The left and right replica cauliflower ears are modeled from a mold of an athlete's cauliflower ears, and suitably resized and shaped to be worn over the ears of the wearer. In another embodiment, the supporting means comprises a flexible head band, and the left and right replica cauliflower ears are fastened to the left and right arms, respectively, of the head band, and wherein the left and right arms am configured to at least partially wrap around a wearer's head.

The present disclosure relates to an apparatus and method for wearing repilca ears.

### BACKGROUND

In mixed martial arts (MMA), wrestling, rugby, boxing and other contact sports, athletes may sustain injuries which may become scarred when the injuries heal. More particularly, athletes in contact sports such as MMK wrestling, rugby and boxing may sustain injuries to the ears resulting in hematoma auris, perichondrial hematoma, or traumatic auricular hematoma, more commonly known as “cauliflower” ears. These cauliflower ears are often unique and distinctive for each athlete, and are often considered by the athletes and their fans to be badges of honor.

### SUMMARY

The present disclosure relates to an apparatus and method for wearing replica “cauliflower” ears which are modeled from the ears of an athlete in MMA, wrestling, or other contact sports.

In an aspect, there is provided an apparatus for wearing replica cauliflower ears, comprising: support means adapted to support left and right replica cauliflower ears on a wearer's head; and fastening means for fastening the left and right replica cauliflower ears, respectively, to the support means; wherein the left and right replica cauliflower ears are modeled from a mold of an athlete's ears, and suitably resized and shaped to be worn over the ears of the wearer.

In an embodiment, at least one of the left and right replica ears shows a condition resulting from hematoma auris, perichondrial hematoma, or traumatic auricular hematoma.

In another embodiment, the supporting means comprises a flexible head band, and the left and right replica cauliflower ears are fastened to the left and right arms, respectively, of the head band, and wherein the left and right arms are configured to at least partially wrap around a wearer's head.

In another aspect there is provided a method for wearing replica cauliflower ears, comprising: molding the left and right cauliflower ears of an athlete; modeling the left and right replica cauliflower ears from the mold of the left and right cauliflower ears of an athlete; and suitably resizing and shaping the replica cauliflower ears to be worn over the ears of a wearer.

In an embodiment, at least one of the left and right replica ears shows a condition resulting from hematoma auris, perichondrial hematoma, or traumatic auricular hematoma.

In an embodiment, the method further comprises: providing a support means adapted to support left and right replica cauliflower ears on a wearer's head; and fastening the left and right replica cauliflower ears, respectively, to the support means.

In this respect, before explaining at least one embodiment in detail, it is to be understood that the present invention is not limited to the specific details of construction and to the arrangements of the components set forth in the following description or illustrated in the drawings. The present invention is capable of other embodiments, and of being practiced and carried out in various ways. Also, it is to be understood that the phraseology and terminology employed herein are for the purpose of description and should not be regarded as limiting.

### BRIEF DESCRIPTION OF THE DRAWINGS

FIGS. 1A-1C show various examples of an apparatus in accordance with several different embodiments;

FIGS. 2A-2C show different views of an apparatus in accordance an embodiment;

FIG. 3 shows a detailed perspective view of an apparatus in accordance with an embodiment;

FIGS. 4A-4C show different views of an apparatus as worn by a wearer in accordance with an embodiment;

FIGS. 5A-5C show a clip system for fastening various articles to the apparatus;

FIG. 6A shows a detailed view of an apparatus as worn by a wearer in accordance with an embodiment; and

FIG. 6B shows a detailed perspective view of another embodiment of the apparatus.

FIGS. 7A-7H show an illustration of another embodiment including a tape band accessory.

FIGS. 8A-8I show a perspective view of another embodiment of the apparatus from various viewing angles.

### DETAILED DESCRIPTION

As noted above, the present disclosure relates to an apparatus and method for wearing replica “cauliflower” ears which are modeled from the ears of an athlete in MMA, wrestling, rugby, boxing or other contact sports.

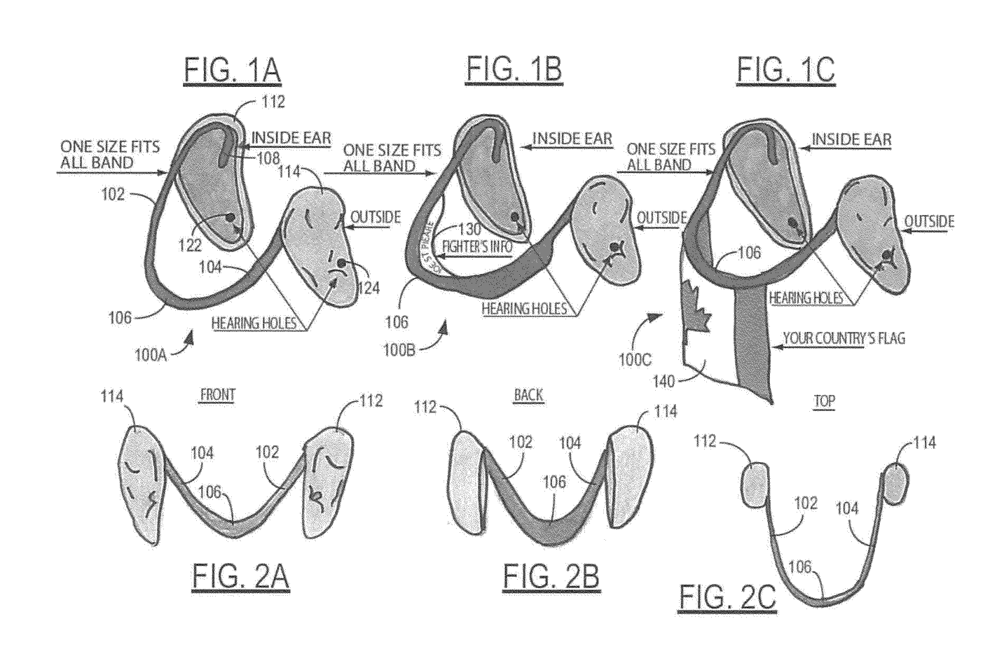

As shown in FIG. 1A, in accordance with an embodiment, apparatus 100A comprises a supporting means, such as a flexible bead band with a left arm 102, a right arm 104, and a supporting arch 108. The left and right arms 102, 104 are adapted to at least partially wrap around a wearer's bead, with the supporting arch 106 being suitably positioned and oriented to keep the left and right arms 102, 104 in position. Each of the left and right arms 102, 104 includes fastening means, such as a fastener 108, for fastening a left replica ear 112 and a right replica ear 114 to apparatus 100. The left and right replica cauliflower ears 112, 114 are modeled from a mold of an athlete's ears to feature the unique and distinctive cauliflower ears of the athlete. The left and right replica cauliflower ears 112, 114 are suitably sized and shaped to be worn over the ears of a wearer, and to give the appearance of the wearer also having cauliflower ears to publicly show support.

In an embodiment, the left and right replica cauliflower ears 112, 114 include orifices 122, 124 to allow ventilation and to allow sounds to better enter the ears of the wearer. Alternatively, for fans who find the noise too loud at matches or events, the left and right replica cauliflower ears 112, 114 do not include orifices 122, 124, and may in fact include noise suppression material if desired. Alternatively, the left and right replica cauliflower ears 112, 114 could also include earphones, such that the wearer can listen to event commentators. In addition to hearing event commentary, the earphones could provide pre-recorded quotes from an athlete that may be triggered by a tap to the replica cauliflower ears 112, 114 or by pressing a button or flipping a switch.

Now referring to FIG. 1B, in another embodiment, apparatus 100B includes all of the features of apparatus 100A of FIG. 1A, but also further includes a display area 130 forming a part of the supporting arch 108. Display area 130 may be used to attach a label and/or display directly printed or engraved text and/or graphics. The text and or graphics as displayed in display area 130 may include information on the athlete, such as the athlete's hometown, tale of the tape, and athlete's information including name, age, height, weight, reach, and titles and honors won (e.g. hall of fame) or currently held by the athlete. In team sports such as rugby, the player's jersey number may also be placed in the display area, together with team colors and other identifying visual features. The athlete's signature may also be provided inside or outside the supporting arch area to provide “limited edition” replica cauliflower ears. If the apparatus 100B is sponsored, suitable advertising may also be placed in the display area 130.

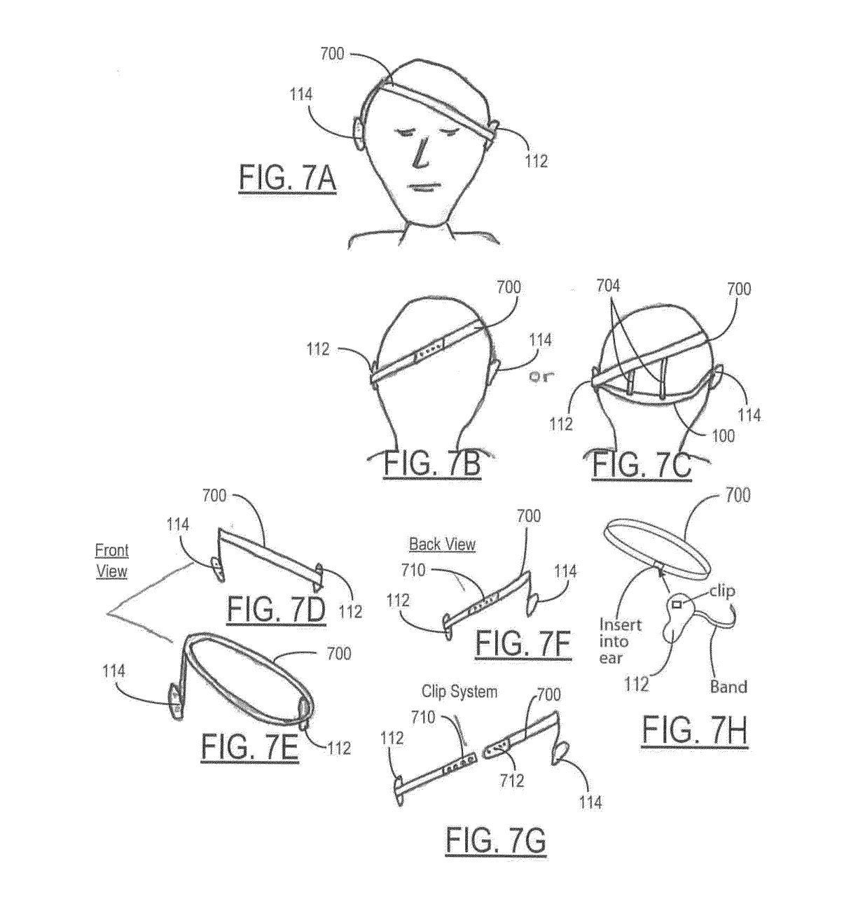

Now referring to FIG. 1C, shown is another illustrative embodiment 100C in which the supporting arch 106 further includes a clip system for fastening an article to the headband with one or more dip fasteners. For example, the article to be clipped may be a flag, or a visual representation of a title belt, for example, won or currently held by the athlete. The clip system is described in more detail further below, with reference to FIG. 5B.

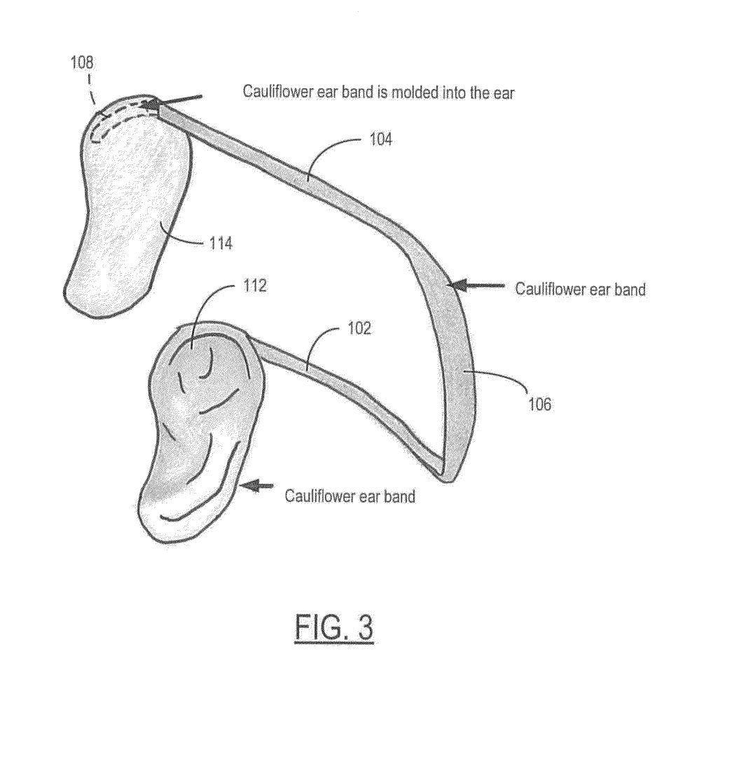

Now referring to FIGS. 2A-2C, shown are an illustrative front view (FIG. 2A), back view (FIG. 2B), and top view (FIG. 2C) of the apparatus 100A of FIG. 1A.

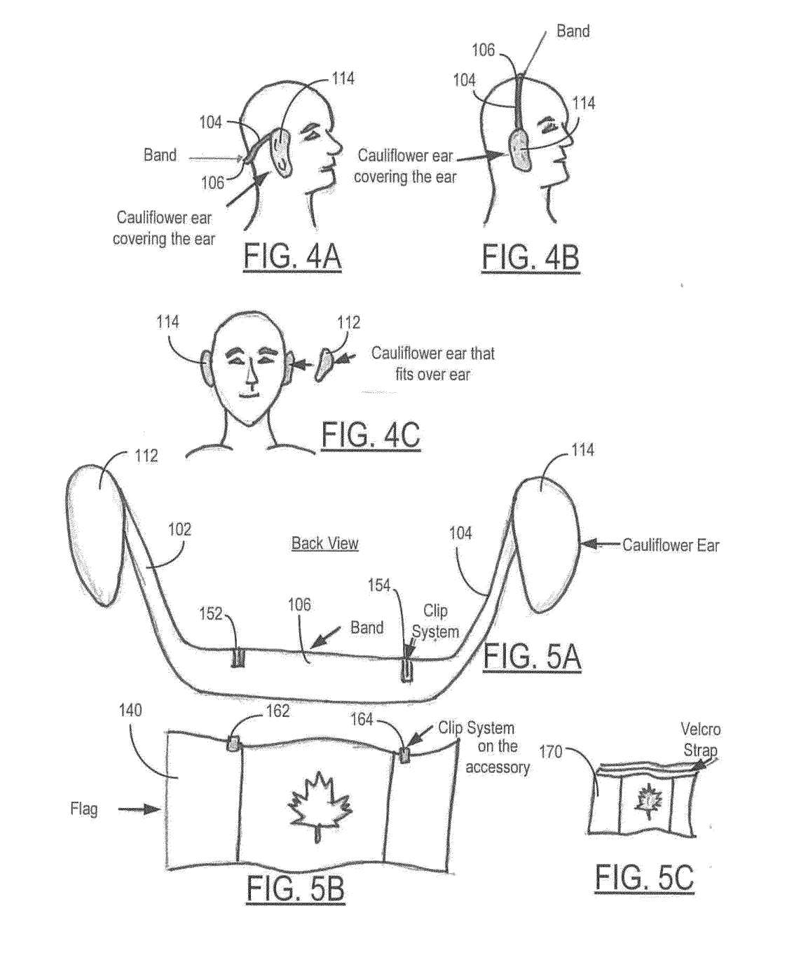

Now referring to FIG. 3, shown is an illustrative example of a fastening means, such as fastener 108, for fastening the left and right replica cauliflower ears 112, 114 to the left and right arms 102, 104 respectively. In an embodiment fastener 108 may, for example, be molded directly into the left and right replica cauliflower ears 112, 114. Fastener 108 may be chemically bonded with a suitable adhesive, or mechanically fastened by means of a fastener such, as a screw or flexible clip. Alternatively, left and right replica cauliflower ears 112, 114 may be removably fastened via fastener 108 such that the wearer can switch the replica cauliflower ears 112, 114 to show support for different athletes for different matches that the wearer may attend. If the it will be appreciated that virtually any fastening means suitable for fastening the left and right replica cauliflower ears 112, 114 may be used.

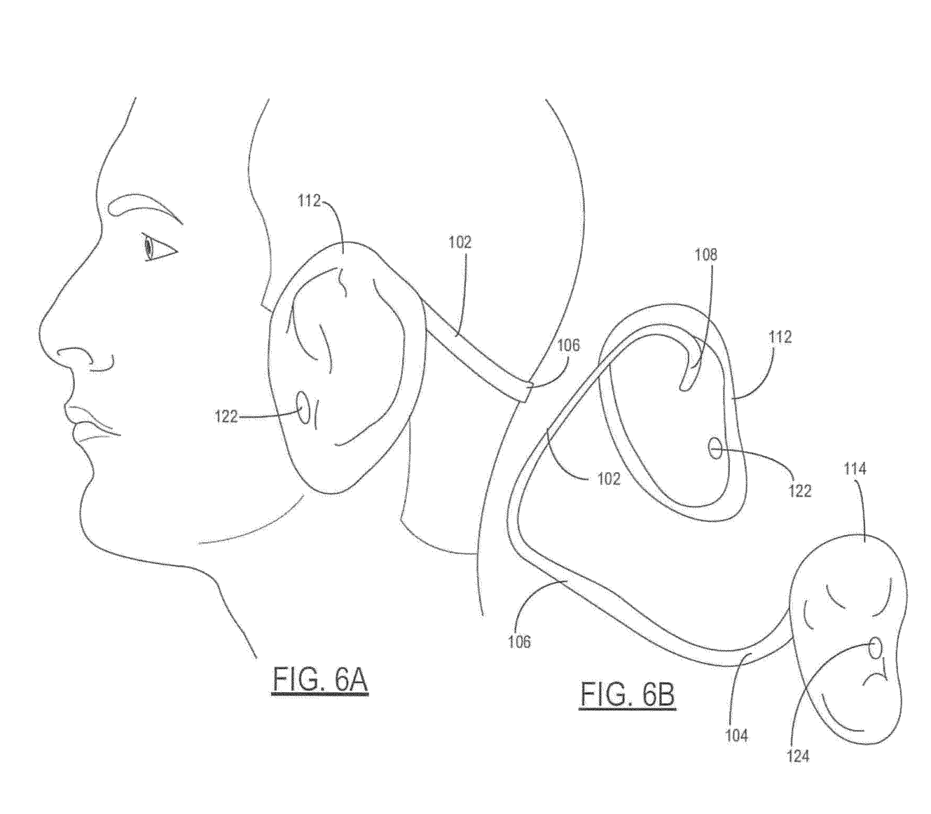

Now referring to FIG. 4A, in an embodiment, apparatus 100 may be worn with let arm 102 (hidden from view) and right arm 104 wrapped around the back of a wearer's head. In this case, supporting arch 106 is oriented towards the back of the wearer's head. Alternatively, FIG. 4B shows apparatus 100 worn with left arm (hidden from view) and right arm 104 wrapped around the top of a wearer's head, in which case supporting arch 108 is positioned at or near the top of the wearer's head.

Now referring to FIG. 4C, shown is another embodiment in which the left and right replica cauliflower ears 112, 114 are wearable directly over the ears of a wearer, without the need to provide a head band to wrap around a portion of the head.

Now referring to FIG. 5A, shown is a back view of an apparatus 100 which now includes an accessory fastening system 152, 154 for fastening items or articles such as a flag 140 shown in FIG. 5B. The clip system may include corresponding clips 182, 184 on flag 140 to match accessory fastening system 152, 154. Alternatively, a hook and loop fastening means may be used to attach an article 170 to apparatus 100, as shown in FIG. 5C. With the present accessory fastening system 152, 154, the articles are completely interchangeable, such that different flags, title holder belts, etc, may be fastened to the apparatus 100.

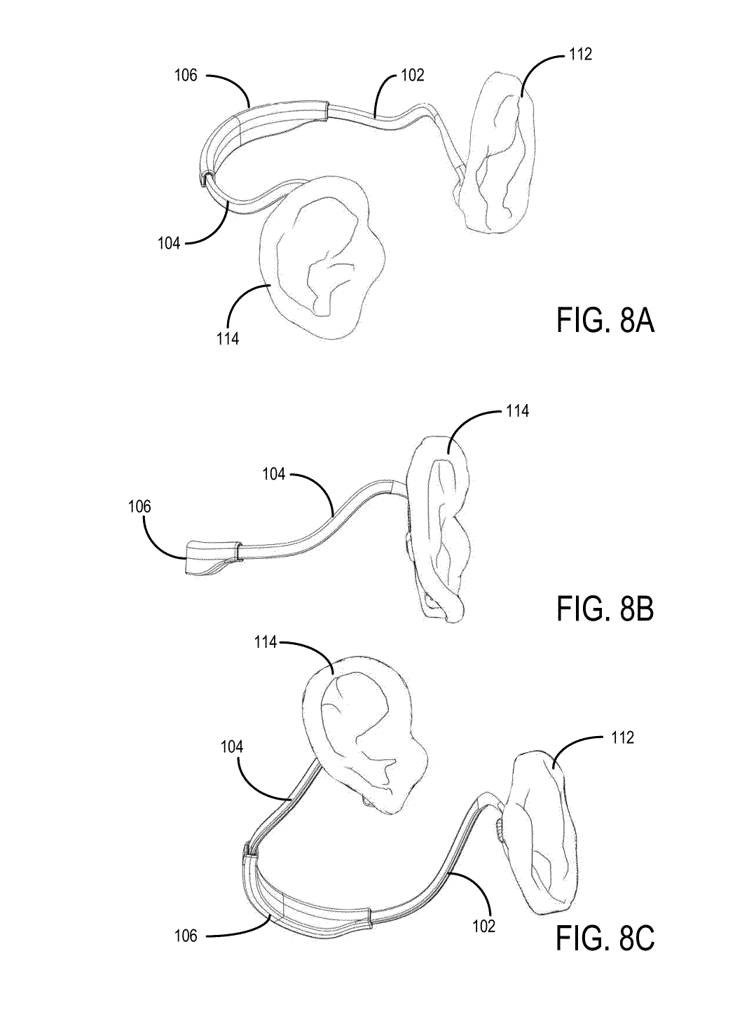

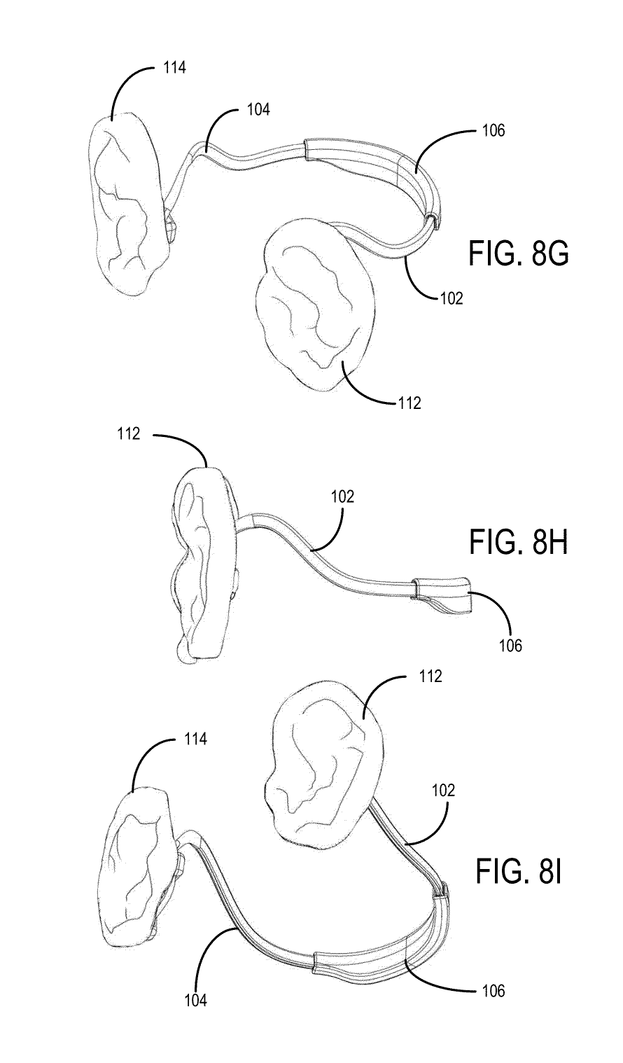

Now referring to FIG. 8A, shown is a side view of an apparatus 100 as worn by a wearer. As shown, in this embodiment, apparatus 100 is worn behind the wearer's head, FIG. 6B shows a more detailed perspective view of apparatus 100 in accordance with an embodiment.

Preferably, the replica cauliflower ears 112, 114 may be made from a suitable, flexible material, such as rubber, silicone or plastic, and modeled from an actual mold of an athlete's years. The replica cauliflower ears 112, 114 are preferably larger than an adult male's ears to fully cover the ears. However, the replica cauliflower ears 112, 114 may also be sized to be smaller for a more customized fit. The replica cauliflower ears 112, 114 may also be significantly larger than the ears of a wearer to make them more prominent.

In an embodiment, earrings may be provided as accessories for the replica cauliflower ears 112, 114 if the athlete wears them.

In another embodiment, the replica cauliflower ears 112, 114 may also include replica hair, shading and color to closely match the actual ears of an athlete. Other features may be provided on the replica cauliflower ears 112, 114 including simulated bruised or bloody ears, for example.

In another embodiment, the replica cauliflower ears 112, 114 may be attached to other types of supporting means, such as a hat or other head covering, or glasses including sunglasses. Limited edition wigs can also be attached to the supporting means to replicate a signature haircut of an athlete. The replica cauliflower ears 112, 114 may also be connected together with string so that they are not easily lost.

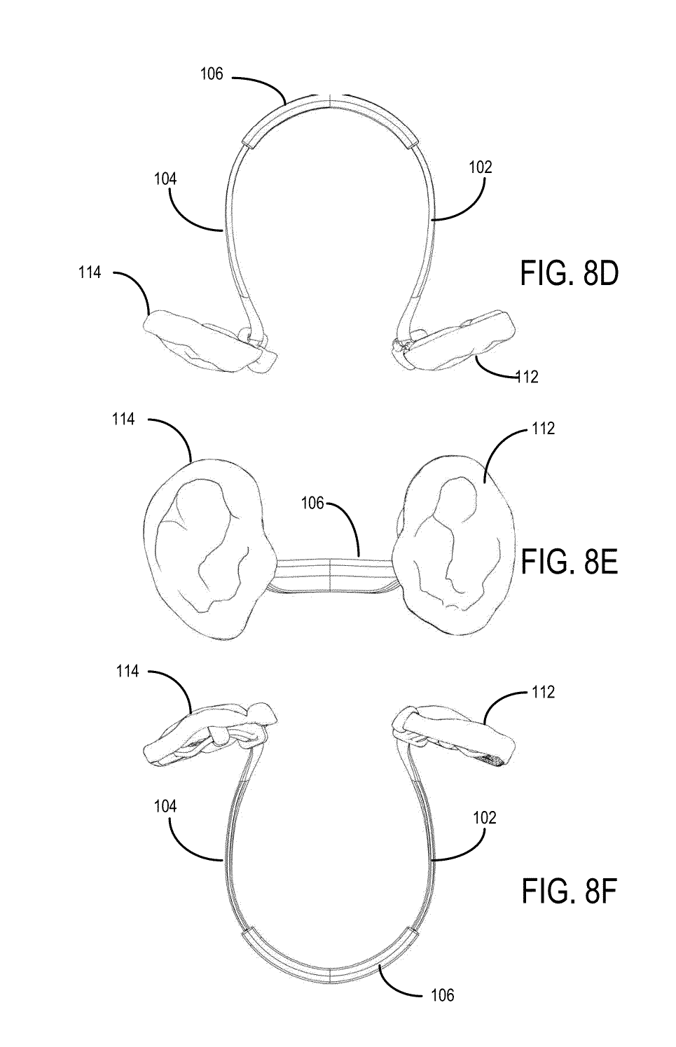

In another embodiment, as shown in FIGS. 7A-7H, the replica cauliflower ears 112, 114 further include a tape band accessory 700 that rugby players use to tape down their cauliflower ears. In an embodiment, the tape band accessory 700 may be clipped on to the head band 100. Alternatively, the tape band accessory may be used as a hat strap (e.g. as shown in FIGS. 7A-7C) with the replica cauliflower ears 112, 114 attached, with the tape band accessory 700 at the back being clipped together with cooperating clip system 710, 712, like a baseball hat (e.g. as shown best in FIGS. 7B, 7F and 7G), to fit all sized heads. As shown in FIG. 7H, a cauliflower ear 112 may be fastened to the tape band accessory to keep the tape band accessory in position. One or more straps 704 (as shown in FIG. 7C) may also secure the tape band accessory 700 and head band 100 together.

In another embodiment, the replica cauliflower ears 112, 114 may be insulated such that they can be used as ear muffs or ear warmers in cold weather.

In another embodiment, the replica cauliflower ears 112, 114 may be used as game console headgear to wear when playing contact sport games such as MMA, wrestling, rugby, boxing, or virtually any other game requiring or providing support for headphones. In this case, apparatus 100 may further include a microphone such that the wearer can also speak in addition to listening via ear phones.

Now referring to FIGS. 8A-8I, shown are illustrative views of another embodiment; of the apparatus from various viewing angles. FIG. 8A shows an apparatus 100 with supporting arch 106 which adjustably supports left arm 102 and right arm 104. By sliding a portion of the left arm 102 and right arm 104 into or out of the supporting arch 108, the apparatus may be adjustable to accommodate the heads of different wearers. Replica cauliflower ears 112, 114 may be adjustably mounted onto the ends of the left arm 102 and right arm 104, respectively, such that the replica cauliflower ears may be suitably adjusted to cover the ears of a wearer. The replica cauliflower ears 112, 114 are also detachable, and replaceable with customized replica cauliflower ears 112, 114 molded from the ears of another prominent athlete.

FIGS. 8B to 8I show views of the apparatus 100 of FIG. 8A from different angles, and illustrates a curve in the left, arm 102 and right arm 104 to better position the replica cauliflower ears 112, 114 in various angles on the head of the wearer. By allowing the apparatus 100 to be adjustable, one apparatus size may be suitable for a wide range of wearers.

Thus, in an aspect, there is provided an apparatus for wearing replica cauliflower ears which comprises a supporting means to support left and right replica cauliflower ears on a wearer's head. For example, the supporting means may be a flexible bead band with left and rights arms adapted to at least partially wrap around a wearer's head. Each of the left and right arms includes fastening means for fastening left and right replica cauliflower ears, respectively, which are modeled from a mold of an athlete's ears. The left and right replica cauliflower ears are modeled to feature the distinctive cauliflower ears of an athlete, and are suitably resized and shaped to be worn over the ears of a wearer.

In another aspect, there is provided a method of wearing replica cauliflower ears comprising molding the left and right ears of an athlete; modeling the left and right replica cauliflower ears from the mold, and suitably resizing the replica cauliflower ears to be worn over the ears of a wearer. In an embodiment, the left and right replica cauliflower ears are fastened to the left and right arms, respectively, of a flexible head band, and the left and right arms are configured to at least partially wrap around a wearer's head.

In another aspect, the flexible head band includes a supporting arch portion adjoining the left and right arms, and the supporting arch includes space for identifying the athlete from whom the replica cauliflower ears are molded. The replica cauliflower ears of a prominent athlete may then be worn by fans seeking to show support for the athlete, for example at an event or match.

In another aspect, the supporting arch area of the flexible headband further includes a clip system for fastening an article to the headband with one or more clip fasteners. For example, the article to be clipped on to the headband may be a flag, or a visual representation of a title belter honor (e.g. hall of fame) won of currently held by the athlete. The athlete's signature may also be provided inside or outside the supporting arch area to provide “limited edition” replica cauliflower ears.

Thus, in one aspect, there is provided an apparatus for wearing replica ears, comprising: support means adapted to support left and right replica ears on a wearer's head; and fastening means for fastening the left and right replica ears, respectively, to the support means; wherein the left and right replica ears are modeled from a mold of an athlete's ears, and suitably resized and shaped to be worn over the ears of the wearer.

In an embodiment, at least one of the left and right replica ears shows a condition resulting from hematoma auris, perichondrial hematoma, or traumatic auricular hematoma (cauliflower ears).

In another embodiment, the supporting means comprises a flexible head band, and the left and right replica cauliflower ears are fastened to the left and right arms, respectively, of the flexible head band, and wherein the left and right arms are configured to at least partially wrap around a wearer's head.

In another embodiment, the supporting means comprises a supporting arch, and left and right arms extending from the supporting arch, the left and right arms having a curve to better position the replica cauliflower ears in various angles on the wearer's head.

In another embodiment, the left and right arms are adjustable relative to the supporting arch, such that the size of the apparatus can fee adjusted to a wearer's head.

In another embodiment, the left and right replica ears are removable, and replaceable by another set of left and right replica ears molded from another athlete's ears.

In another embodiment, the support means further comprises a clip system for fastening an article to the apparatus.

In another embodiment, the article is one of a flag or a visual representation of a title belt corresponding to an athlete from whose ears the left and right replica ears are molded.

In another embodiment, the support means further comprises a display area to which a label, text or graphics may be attached, directly printed on, or engraved.

In another embodiment, the label, text or graphics comprises one or more of information on the athlete, the player's jersey number, the player's team colors, or advertising from a sponsor.

In another aspect, there is provided a method of wearing replica ears, comprising: providing support means adapted to support left and right replica ears on a wearer's head; and providing fastening means for fastening the left and right replica ears, respectively, to the support means; wherein the left and right replica ears are modeled from a mold of an athlete's ears, and suitably resized and shaped to fee worn over the ears of the wearer.

In an embodiment, the method further comprises showing on at least one of the left and right replica ears a condition resulting from hematoma auris, perichondrial hematoma, or traumatic auricular hematoma (cauliflower ears).

In another embodiment, the method further comprises providing as the supporting means a flexible head band with left and right arms, the left and right replica cauliflower ears being fastened to the left and right arms, respectively, of the flexible head band, and wherein the left and right arms are configured to at least partially wrap around a wearers head.

In another embodiment, the method further comprises providing as the supporting means a supporting arch, and left and right arms extending from the supporting arch, the left and right arms having a curve to better position the replica cauliflower ears in various angles on the wearer's head.

In another embodiment, the method further comprises providing left and right arms which are adjustable relative to the supporting arch, such that the size of the apparatus can be adjusted to a wearer's head.

In another embodiment, the method further comprises providing left and right replica ears which are removable, and replaceable by another set of left and right replica ears molded from another athlete's ears.

In another embodiment, the method further comprises providing on the support means a clip system for fastening an article.

In another embodiment, the method further comprises providing as the article one of a flag or a visual representation of a title belt corresponding to an athlete from whose ears the left and right replica ears are molded.

In another embodiment, the method further comprises providing on the support means a display area to which a label, text or graphics may be attached, directly printed on, or engraved.

In another embodiment, the method further comprises providing as the label, text or graphics one or more of information on the athlete, the players jersey number, the player's team colors, or advertising from a sponsor.

While the above description provides examples of one or more systems and methods, it will be appreciated that other systems and methods may be within the scope of the present description as interpreted by one of skill in the art.

What is claimed is:

1. An apparatus for wearing replica ears, comprising:
support means adapted to support left and right replica ears on a wearer's head; and
fastening means for fastening the left and right replica ears, respectively, to the support means;
wherein the left and right replica ears are modeled from a mold of an athlete's ears, and suitably resized and shaped to be worn over the ears of the wearer.

2. The apparatus of claim 1, wherein at least one of the let and right replica ears shows a condition resulting from hematoma auris, perichondrial hematoma, or traumatic auricular hematoma (cauliflower ears).

3. The apparatus of claim 2, wherein the supporting means comprises a flexible head, band, and the left and right replica cauliflower ears are fastened to the left and right arms, respectively, of the flexible head band, and wherein the let and right arms are configured to at least partially wrap around a wearer's head,

4. The apparatus of claim 3, wherein the supporting means comprises a supporting arch, and left and right arms extending from the supporting arch, the left and right arms having a curve to better position the replica cauliflower ears in various angles on the wearer's head.

5. The apparatus of claim 4, wherein the left and right arms are adjustable relative to the supporting arch, such that the -size of the apparatus can be adjusted to a wearer's head.

6. The apparatus of claim 1, wherein tie left and right replica ears are removable, and replaceable by another set of let and right replica ears molded from another athlete's ears.

7. The apparatus of claim 1, wherein the support means further comprises a clip system for fastening an article to the apparatus.

8. The apparatus of claim 7, wherein the article is one of a flag or a visual representation of a title belt corresponding to an athlete from whose ears the left and right replica ears are molded.,

9. The apparatus of claim 1, wherein the support means further comprises a display area to which a label, text or graphics may be attached, directly printed on, or engraved,

10. The apparatus of claim 9, Wherein the label, text or graphics comprises one or more of information on the athlete, the player's jersey number, the player's team colors, or advertising from a sponsor.

11. An method of wearing replica ears, comprising;
providing support means adapted to support, left and; right replica ears on a wearer's head; and
providing fastening means for fastening the left and right replica ears, respectively to the support means;
wherein the left and right replica ears are modeled from a mold of an athlete's ears, and suitably resized and shaped to be worn over the ears of the wearer.

12. The method of claim 11, further comprising showing on at least one of the left and right replica ears a condition resulting from hematoma auris, perichondrial hematoma, or traumatic auricular hematoma (cauliflower ears).

13. The method of claim 12, further comprising providing as the supporting means a flexible head band with left and right arms, the left, and right replica cauliflower ears being fastened to the left and right arms, respectively, of the flexible head band, and wherein the left and right arms are configured to at least partially wrap around a wearer's head,

14. The method of claim 13, further comprising providing as the supporting means a supporting arch, and left and right, arms extending from the supporting arch, the left and right arms having a curve to better position the replica cauliflower ears in various angles on the wearer's head.

15. The method of claim 14, further comprising providing left and right arms which are adjustable relative to the supporting arch, such that the size of the apparatus can be adjusted to a wearer's head.

16. The method of claim 11, further comprising providing left and right replica ears which are removable, and replaceable by another set of left and right replica ears molded from another athlete's ears.

17. The method of claim 11, further comprising providing on the support means a dip system for fastening an article.

18. The method of claim 17, further comprising providing as the article one of a flag or a visual representation of a title belt corresponding to an athlete from whose ears the left and right replica ears are molded.

19. The method of claim 11, further comprising providing on the support means a display area to which a label, text or graphics may be attached, directly printed on, or engraved.

20. The method of claim 18, further comprising providing as the label, text or graphics one or more of information on the athlete, the player's jersey number, the player's team colors, or advertising from a sponsor.
